(19) United States
(12) Patent Application Publication
(10) Pub. No.: US 2025/0222033 A1
(43) Pub. Date: Jul. 10, 2025

(54) THERAPEUTIC COMPOSITION FOR THE TREATMENT OF DRY EYE DISEASE AND PROMOTION OF OCULAR HEALTH

(71) Applicant: PReye, LLC, Golden, CO (US)

(72) Inventor: Melinda ORourke, Golden, CO (US)

(73) Assignee: PReye, LLC, Golden, CO (US)

(21) Appl. No.: 19/010,962

(22) Filed: Jan. 6, 2025

Related U.S. Application Data
(60) Provisional application No. 63/619,649, filed on Jan. 10, 2024.

(51) Int. Cl. A61K 35/19; A61K 8/36; A61K 8/64; A61K 8/67; A61K 8/73; A61K 8/96; A61K 31/07; A61K 31/192; A61K 31/197; A61K 31/375; A61K 31/714; A61K 31/728; A61K 38/13; A61Q 7/00

(52) U.S. Cl. CPC A61K 35/19; A61K 8/36; A61K 8/64; A61K 8/671; A61K 8/673; A61K 8/676; A61K 8/735; A61K 8/96; A61K 31/07; A61K 31/192; A61K 31/197; A61K 31/375; A61K 31/714; A61K 31/728; A61K 38/13; A61Q 7/00

(57) ABSTRACT

A composition for treating dry eye disease and/or promoting eyelash growth includes at least three compounds selected from the group consisting of platelet rich plasma (PRP), Acetylcysteine, Vitamin C (L-Ascorbic Acid), Vitamin B12, cyclosporine A, ferulic acid, Vitamin A, and hyaluronic acid (HA). A composition for maintaining and promoting ocular health, including, without limitation, Vitamin B12, Vitamin C, Vitamin D, and Vitamin E, Vitamin A, Acetylcysteine, hyaluronic acid, and ferulic acid. In a method for treatment of dry eye disease, for promoting eye lash growth, and for maintaining ocular health, the composition(s) is administered via an eye drop applicator or via a syringe or cannula to the eye of a patient.

### CROSS REFERENCE TO RELATED APPLICATIONS

This application claims priority to and claims the benefit of U.S. Provisional Application No. 63/619,649 filed Jan. 10, 2024, entitled “Therapeutic Composition for The Treatment of Dry Eye Disease and Promotion of Eyelash Growth”, the entire contents of which are hereby expressly incorporated herein by this reference including, without limitation, the specification, claims and abstract, as well as any figures, tables, or drawings thereof.

### TECHNICAL FIELD

The subject matter disclosed herein is generally directed to the field of ophthalmology, and in particular, relates to a therapeutic composition for the treatment of dry eye disease and promotion of eyelash growth, and another composition for the promotion of ocular health.

### BACKGROUND

Dry eye disease (DED) or dysfunctional tear syndrome, also known as keratoconjunctivitis sicca, is a disorder of the tear film and ocular surface of the eye. DED is potentially disabling by impairing vision. DED, as a multifunction disorder, results in visual disturbance, visual performance degradation with regard to sensitivity to contrast or reading speed, for example, and a general loss of productivity and vision-related quality of life.

Millions of people worldwide are affected by DED. While estimates on the prevalence of DED vary greatly, it is estimated that approximately 6.8% of the adult population in the US, or about 16.4 million people have moderate to severe DED. Prevalence increases with age and is higher among women than men, affecting approximately 11.1 million women and 5.3 million men in the U.S. adult population. https://www.sciencedirect.com/science/article/pii/S0002939417302908. The actual incidence of DED is likely much higher as many cases are not diagnosed.

DED symptoms generally have been managed with over-the-counter medications, such as artificial tears, which may offer temporary relief for DED symptoms. Other treatments have included topical antibiotic ointments, for example, bacitracin or erythromycin ointments, oral antibiotics, such as doxycycline, and corticosteroids. One common pharmacological composition currently available for treatment of DED is cyclosporine (Restasis®, Cequa, Veveye). Currently available treatments, however, are often ineffective or variably effective for moderate to severe DED symptoms and further are often time-consuming, expensive, or difficult for a patient to tolerate.

In light of the heterogenous nature of DED and the limitations associated with currently available treatments, there is a need for an improved composition for the treatment of DED that addresses the limitations of currently available treatments. There is a further need for a DED treatment that also may promote eyelash growth and additionally provide vitamin supplements that not only improve DED symptoms but improves eye health.

Further, oxidative damage to the eye caused by UV light affects millions of people worldwide. Ocular surface disease, cataracts, and macular degeneration have all been proven to be caused or made worse by chronic exposure to UV light. Current modalities to prevent UV damage to the eye include wearing sunglass protection with UV blockage, and oral antioxidant vitamins to slow the progression of macular degeneration. However, not everyone has access to sunglasses, and UV rays can still penetrate around protective lenses. Oral antioxidant vitamins will enter the bloodstream including the retinal vasculature to deliver antioxidant protection to the retina and macula. However, the corneal surface is avascular and does not receive direct circulation from the blood supply. Applicant describes herein embodiments of compositions for the promotion of ocular health that overcome the limitations of currently available methods, by delivering antioxidants directly to the corneal surface to aid in the protection of the eye from oxidative damage caused by UV light exposure, essentially a topical sunscreen for the eye.

Citation or identification of any document in this application is not an admission that such a document is available as prior art to the present invention.

### SUMMARY

In one example embodiment, a composition for treating dry eye disease comprises at least three compounds selected from the group consisting of platelet rich plasma (PRP), Acetylcysteine, Vitamin C (L-Ascorbic Acid), Vitamin B12, saline, a stabilizer, and hyaluronic acid. In one example embodiment, the composition further includes cyclosporine A and/or Vitamin A. The stabilizer may comprise ferulic acid.

In one example embodiment, a composition for promoting eye lash growth comprises at least three compounds selected from the group consisting of platelet rich plasma (PRP), Acetylcysteine, Vitamin C (L-Ascorbic Acid), saline, stabilizer, and hyaluronic acid (HA), In one example embodiment, the composition further includes Vitamin A, Vitamin B12, ferulic acid, and/or cyclosporine A.

In one example embodiment, a composition for treating dry eye disease comprises at least three compounds selected from the group consisting of platelet rich plasma (PRP), Acetylcysteine, Vitamin C (L-Ascorbic Acid), cyclosporine A, saline, stabilizer such as ferulic acid, hyaluronic acid (HA), Vitamin A, Vitamin B12, Vitamin D, and Vitamin E.

In one example embodiment, a composition for treating dry eye disease comprises administering 100% pure platelet rich plasma (PRP), with or without saline or hyaluronic acid (HA).

In one example embodiment, a composition for promoting eye lash growth comprises at least three compounds selected from the group consisting of platelet rich plasma (PRP), Acetylcysteine, Vitamin C (L-Ascorbic Acid), cyclosporine A, saline, stabilizer such as ferulic acid, hyaluronic acid (HA), Vitamin A, Vitamin B12, Vitamin D, and Vitamin E.

In one example embodiment, a composition for promoting ocular health comprises at least three compounds selected from the group consisting of Acetylcysteine, Vitamin C (L-Ascorbic Acid), ferulic acid, hyaluronic acid (HA), saline, Vitamin A, Vitamin B12, Vitamin D, and Vitamin E.

In one example embodiment, a method for treating dry eye disease comprises administering to a subject a composition comprising at least three compounds selected from the group consisting of platelet rich plasma (PRP), Acetylcysteine, Vitamin C (L-Ascorbic Acid), Vitamin B12, saline, stabilizer such as ferulic acid, hyaluronic acid (HA), and cyclosporine A.

In one example embodiment, a method for promoting eye lash growth comprises administering to a subject a composition comprising at least three compounds selected from the group consisting of platelet rich plasma (PRP), Acetylcysteine, Vitamin C (L-Ascorbic Acid), Vitamin B12, saline, stabilizer, hyaluronic acid (HA), and cyclosporine A.

In one example embodiment, a method for treating dry eye disease comprises administering to a subject a composition comprising at least three compounds selected from the group consisting of platelet rich plasma (PRP), Acetylcysteine, Vitamin C (L-Ascorbic Acid), cyclosporine A, saline, stabilizer such as ferulic acid, hyaluronic acid (HA), Vitamin A, Vitamin B12, Vitamin D, and Vitamin E.

In one example embodiment, a method for promoting eye lash growth comprising administering to a subject a composition comprising at least three compounds selected from the group consisting of platelet rich plasma (PRP), Acetylcysteine, Vitamin C (L-Ascorbic Acid), cyclosporine A, saline, ferulic acid, hyaluronic acid (HA), Vitamin A, Vitamin B12, Vitamin D, and Vitamin E.

In one example embodiment, a method for promoting ocular health comprises administering to a subject a composition comprising at least three compounds selected from the group consisting of Acetylcysteine, Vitamin C (L-Ascorbic Acid), saline, ferulic acid, Vitamin A, Vitamin B12, Vitamin D, and Vitamin E.

In one example embodiment, a method for treating dry eye disease comprises administering to a subject only platelet rich plasma (PRP), with or without saline, or hyaluronic acid (HA).

In one example embodiment, a composition for eye health comprises at least three compounds selected from the group consisting of Acetylcysteine, Vitamin C (L-Ascorbic Acid), Vitamin B12, saline, stabilizer, and hyaluronic acid. In one example embodiment, the composition further includes cyclosporine A and/or Vitamin A. Ferulic acid may be used as a stabilizer.

In one example embodiment, a composition for eye health comprises at least three compounds selected from the group consisting of Acetylcysteine, Vitamin C (L-Ascorbic Acid), saline, stabilizer, and hyaluronic acid (HA), In one example embodiment, the composition further includes Vitamin A, Vitamin B12, and/or cyclosporine A.

In one example embodiment, a composition for eye health comprises at least three compounds selected from the group consisting of Acetylcysteine, Vitamin C (L-Ascorbic Acid), cyclosporine A, ferulic acid, hyaluronic acid (HA), Vitamin A, Vitamin B12, Vitamin D, and Vitamin E.

In one example embodiment, a composition for eye health comprises at least three compounds selected from the group consisting of Acetylcysteine, Vitamin C (L-Ascorbic Acid), saline, stabilizer such as ferulic acid, hyaluronic acid (HA), Vitamin A, Vitamin B12, Vitamin D, and Vitamin E.

In one example embodiment, a method for promoting eye health comprises administering to a subject a composition comprising at least three compounds selected from the group consisting of Acetylcysteine, Vitamin C (L-Ascorbic Acid), Vitamin B12, saline, stabilizer, hyaluronic acid (HA), and cyclosporine A.

In one example embodiment, a method for promoting eye health comprises administering to a subject a composition comprising at least three compounds selected from the group consisting of Acetylcysteine, Vitamin C (L-Ascorbic Acid), cyclosporine A, saline, stabilizer such as ferulic acid, hyaluronic acid (HA), Vitamin A, Vitamin B12, Vitamin D, and Vitamin E.

In one example embodiment, a method for promoting ocular health comprises administering to a subject a composition comprising at least three compounds selected from the group consisting of Acetylcysteine, Vitamin C (L-Ascorbic Acid), saline, stabilizer, Vitamin A, Vitamin B12, Vitamin D, and Vitamin E.

These and other aspects, objects, features, and advantages of the example embodiments will become apparent to those having ordinary skill in the art upon consideration of the following detailed description of example embodiments.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance, or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is whole blood, used to create the PRP.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Dry Eye Disease (DED) affects millions of people worldwide and is found to be more prevalent in women. Current therapeutic treatments for DED typically address inflammation of the ocular surface. A patient suffering from DED typically experiences a combination of abnormalities in the lipid layer of the tear film, tear hyperosmolarity, and/or tear film instability. Common therapeutic treatments tend to focus on reducing inflammation; for example, cyclosporine (Restastis®) is often prescribed to DED patients, while other therapies may involve the use of corticosteroids, topical or oral antibiotics, and artificial tears. These treatments vary in effectiveness for patients suffering from moderate to severe DED symptoms.

Applicant describes and demonstrates embodiments of compositions for the treatment of DED, for promotion of eyelash growth, and for promotion of general ocular health that overcome the limitations of currently available treatments. As described herein, the therapeutic treatments are supported by efficacy, mechanism, and safety.

Antioxidant Vitamins provide protection against the damaging effects of UV light from sun exposure. When UV light is absorbed into human tissue it produces free oxygen radicals that cause damage to the integrity of the cell and DNA. Only 2 organs in the human body are directly exposed to UV light, the skin and the eyes. In the skin, the result is premature aging, skin cancer, and cell death. In the eye, UV light ages all the structures. Ocular surface damage, cataracts and macular degeneration are all possible effects from UV exposure and can ultimately lead to decreased vision, and blindness.

When antioxidant vitamins are present in the tissue, they neutralize the free oxygen radicals and minimize damage. Antioxidants that penetrate the skin barrier are used commonly to prevent aging in the skin. The AREDS (Age Related Eye Disease Study), was an extensive study that proved the benefits of oral antioxidants in the prevention of macular degeneration progression, and the “AREDS” formula of oral antioxidant vitamin supplements are recommended universally in the Ophthalmology community for the prevention of macular degeneration.

Ascorbic Acid (Vitamin C) is a powerful antioxidant that is present naturally in significantly higher levels in human tears compared to the blood, evidence that was proven by a study done by this applicant and published in the Archives of Ophthalmology and presented at Oxford University. The human eye actively pumps Vitamin C into the tears, suggesting an evolutionary benefit to Vitamin C in the tears, presumably due to its protective antioxidant effect against UV damage.

Stabilizers are used to preserve the efficacy of Vitamin C. One such stabilizer, ferulic acid, is a plant-based antioxidant that has been used in anti-aging skin care products and is naturally found in a variety of foods. Ferulic acid, when combined in solution with Vitamin C, may stabilize Vitamin C up to 90%, as Vitamin C may tend to destabilize fairy quickly when in solution. Ferulic acid can also contribute to the overall antioxidant effect and work synergistically with Vitamin C to protect the ocular surface. Ferulic acid may be dissolved in propylene glycol to further aid in the solubility of ferulic acid and then filtered. Other stabilizers may be used without departing from the scope of the disclosure. The terms “such as” when used in combination with ferulic acid are not meant to limit the range of stabilizers that may be used.

Endogenous Glutathione is a critical molecule in the protection against the cytotoxic effects of UV light. Glutathione exists naturally in an unusually high concentration in the lens of the eye where it functions as an antioxidant vital for maintenance of the lens' transparency, to prevent cataracts. Acetylcysteine is converted to Glutathione so it may help to maintain lens clarity by preventing oxidative damage in the lens, and may help to prevent vision loss secondary to retinal degenerative diseases, including age-related macular degeneration.

Both Ascorbic Acid and Acetylcysteine have been used independently to treat pathology in the eye including corneal alkali burns, and filamentary keratitis in severe dry eye. But, never have they been combined as a vitamin supplement eyedrop to prevent eye disease related to UV exposure.

Hyaluronic acid is a naturally occurring lubricant in the eye and is commonly used in artificial tears to provide comfort to the ocular surface. The presence of an antioxidant may enhance the production of endogenous hyaluronic acid and decrease the breakdown of Hyaluronic acid, optimizing the hyaluronic acid on the ocular surface.

Other vitamins with scientific evidence for being advantageous to Ocular Health include Vitamin B12, Vitamin A, Vitamin D, and Vitamin E, as well as Cyclosporine A.

In a further aspect, an Ophthalmology prep kit for PRP eyedrops is contemplated, to streamline the processes to be described below regarding treatment at Ophthalmology clinics. This Ophthalmology Prep kit will include, but may not be limited to, a PRP processing kit provided by a PRP processing company, any appropriate blood draw equipment including tourniquet, butterfly needle, alcohol pads, etc., the 20cc syringe necessary to draw off the PRP after centrifuge in which the eyedrops will be prepared, a vial of approximately 10cc of either saline or the eye drop (DED) solution containing the HA and Vitamin supplements to be added to the PRP, a 12 pack of sterile 3cc eyedropper bottles, a carrying pack with labels to give the patient to take home and store in the freezer, and possibly a cold pack made to keep eyedrops cold outside refrigeration for patient compliance, and/or any other supplies deemed necessary that is specific to the use of PRP and eyedrops, to allow for convenience and efficiency in the Ophthalmology setting.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Compositions

The present invention therefore provides novel therapeutic compositions for the treatment of dry eye disease, for the promotion of eyelash growth, and for the promotion of general ocular health that overcome the disadvantages of known treatments for DED.

In certain aspects, the composition combines three or more compounds selected from cyclosporine A, Platelet Rich Plasma (PRP), Acetylcysteine, L-Ascorbic Acid (Vitamin C), Vitamin B12, ferulic acid, saline and hyaluronic acid (HA). The invention further was shown to be beneficial in the promotion of eyelash growth while simultaneously alleviating the symptoms of DED.

In one aspect, a composition for treating dry eyes and promoting lash growth combines three or more compounds selected from cyclosporine A, Platelet Rich Plasma (PRP), Acetylcysteine, saline, hyaluronic acid (HA), L-Ascorbic Acid (Vitamin C), ferulic acid, Vitamin B12, retinol (Vitamin A), cobalamin (Vitamin B12), calciferol (Vitamin D), and tocopherol or alpha-tocopherol (Vitamin E).

In one aspect, the composition for the promotion of ocular health combines three or more compounds selected from cyclosporine A, Acetylcysteine, L-Ascorbic Acid (Vitamin C), Vitamin B12, saline, ferulic acid, and hyaluronic acid (HA).

In one example embodiment, a composition for the promotion of ocular health combines three or more compounds selected from cyclosporine A, Acetylcysteine, ferulic acid, saline, hyaluronic acid (HA), L-Ascorbic Acid (Vitamin C), Vitamin B12, retinol (Vitamin A), cobalamin (Vitamin B12), calciferol (Vitamin D), and tocopherol or alpha-tocopherol (Vitamin E).

In one example embodiment, a composition for the promotion of ocular health combines three or more compounds selected from Acetylcysteine and/or L-Ascorbic Acid (Vitamin C), retinol (Vitamin A), cobalamin (Vitamin B12), calciferol (Vitamin D), and tocopherol or alpha-tocopherol (Vitamin E).

### Platelet Rich Plasma

Platelet Rich Plasma (PRP) is a well-recognized form of therapy in various medical specialties, particularly in orthopedics and dermatology, to promote healing and regeneration of tissue. Studies have evaluated the effects of PRP drops in patients with dry eye disease and found significant improvements in dry eye symptoms, lacrimal meniscus, conjunctival hyperemia, corneal fluorescein staining and an increase in conjunctival goblet cells. PRP has recently been shown to help with Dry Eye Disease topically, but it can cause irritation if the drops are not properly processed with the appropriate dilutional factors and diluents as this applicant proposes, and by injection around the eye to the meibomian glands and near the lacrimal gland. Autologous serum eye drops are routinely used for dry eyes, but these do not contain platelets. The platelets contain the most important healing components and growth factors in the blood, and without the platelets in autologous serum eye drops, they are much less effective for dry eye therapy when compared to PRP eye drops.

In addition, PRP injections have been shown to increase the survival of hair follicle cells through anti-apoptotic effects and stimulate hair growth by extending the anagen phase of the hair cycle (6) and is commonly used for alopecia and male pattern baldness. Bimatoprost eye drops have been shown to promote eyelash growth, showing that eye drops can access the eyelash hair follicle to promote growth. Considering this evidence that PRP promotes hair growth and the eyelash follicle is accessible through treatment with eye drops, the present invention provides an improved composition for treatment of DED that also promotes eyelash growth. Currently available eye drops, used to treat DED or other eye conditions, do not utilize PRP to promote eyelash growth.

### Cyclosporine

Cyclosporine is proven to be a valuable ophthalmic therapy for Dry Eye Disease through its immunomodulatory actions and regulation of the adaptive immune response, and its mechanism of action is well described in the published literature in the treatment of Dry Eye Disease. Cyclosporine is discussed as an example but not as a limitation. Compositions that do not include Cyclosporine are anticipated and included in the scope of this disclosure.

### Lash Growth

Cyclosporine enhances hair growth through caspase-dependent pathways by retarding anagen-to-catagen phase transition in the hair follicle growth cycle.

### Acetylcysteine

Acetylcysteine is a precursor to glutathione, a powerful antioxidant. Acetylcysteine is more permeable to the cell membrane than glutathione, so topical Acetylcysteine can enter the cell where it converts to Glutathione to provide bioavailability of Glutathione within the cell. The eye is exposed to UV light which results in free oxygen radicals that can damage cell membranes. The presence of an antioxidant can neutralize free oxygen radicals and prevent cell damage due to the UV light. The critical antioxidant power of Acetylcysteine is due to its role as a precursor to glutathione, which is one of the most important naturally occurring antioxidants in the eye. Acetylcysteine results in an elevation of glutathione biosynthesis. Glutathione acts directly as a scavenger of free oxygen radicals. In the cornea, glutathione also plays an important role in maintaining normal hydration level, and in protecting cellular membrane integrity.

The lens of the eye naturally contains a high concentration of reduced glutathione, which contributes to the lens complete transparency as well as to the transparent and refractive properties of the cornea. Acetylcysteine has been used topically in the treatment of corneal wounds, chemical injuries, keratitis, dry eye disease and meibomian gland dysfunction, but never as a vitamin supplement eyedrop to promote eye health.

### Lash Growth:

Acetylcysteine has been shown to promote hair growth in men with alopecia, a general term for hair loss.

### L-Ascorbic Acid (Vitamin C)

Vitamin C is a potent antioxidant that helps reduce oxidative stress and prevent UV damage, and may improve the ocular surface by improving tear breakup time and goblet cell density. Vitamin C also promotes healing by forming and maintaining connective tissue, including collagen in the cornea. Topical Vitamin C eye drops have been used successfully for the treatment of Corneal Alkali burns. Use of compositions that do not include Vitamin C are anticipated and included in this disclosure.

Furthermore, the Applicant's own original research, published in the Archives of Ophthalmology, and presented at Oxford University, showed that Vitamin C levels in human tears are significantly higher than that of plasma, suggesting there is an evolutionary benefit to higher levels of Vitamin C in human tears, be it for its healing benefits, or protective antioxidant effects on the eye.

### Lash Growth:

It has been shown that a derivative of Vitamin C stimulates hair follicle growth in vitro, and it's suggested that ascorbic acid strengthens the connective tissues increasing collagen production, to improve hair growth.

### Ferulic Acid

Ferulic acid, acting as a stabilizer, is a plant-based antioxidant that has been used in anti-aging skin care products and is naturally found in a variety of foods. Ferulic acid, when combined in solution with Vitamin C, may stabilize Vitamin C up to 90%, as Vitamin C may tend to destabilize fairy quickly when in solution. Propylene glycol may be used as a solvent for the ferulic acid. Ferulic acid may be added as a stabilizer but is not required and other stabilizers may be used without departing from the scope of the disclosure.

### Vitamins A, B12, D and E

Vitamins play a critical role to maintain the ocular surface. Vitamins A, C, and E are physiologically present in the ocular surface and are essential for cell differentiation, development, and correct function. Vitamin D and B have a key regulation activity of the immune and nervous systems, respectively, Vitamin deficiencies are frequently associated with both Ocular Surface Disease and Dry Eyes, and systemic supplementation has proved to be beneficial in subgroups of patients with low vitamin intake.

Vitamin A improves the function of the conjunctival goblet cells, increasing mucin expression and promotes corneal and conjunctival wound healing, reducing keratinization (2,3). Multiple studies have shown the efficacy and safety of topical Vitamin A eye drops or ointment in both animal and human models. Specifically, Vitamin A retinol palmitate (VApal) ophthalmic solution (500 IU/mL) was shown to be effective in the treatment of patients with dry eye.

There are several reports suggesting that Vitamin B12 eye drops may improve signs and symptoms of Dry Eye Disease, and Vitamin B12 deficiency has been associated with Sjogren's Syndrome and Neuropathic corneal pain. A recent study showed that increased oxidative stress is present in patients with dry eye compared with those without dry eye, and that treatment with eye drops containing hyaluronic acid 0.15% and vitamin B12 reduces oxidative stress in addition to improving the symptoms of dry eye (30). In addition, 0.05% Vitamin B12 eye drops have been shown to result in corneal nerve regeneration in rats (33). Hyaluronic acid 0.3% and vitamin B12 eye drops effectively decreased dry eye symptoms in menopausal women and improved tear stability and volume (34). Vitamin B12 deficiencies have been associated with Sjogren's Syndrome and Corneal Neuropathic Pain, and symptoms improved with Vitamin B12 supplements (31, 32, 35). Another study showed that Vitamin B12 eye drops can lessen symptoms of dry eye, improve accommodative function and treat vision fatigue caused by VDT (Visual Display Terminals) (36).

### Methods of Use

In one example embodiment, a method for treating dry eye disease comprises administering to a subject a composition comprising at least three compounds selected from the group consisting of platelet rich plasma (PRP), Acetylcysteine, Vitamin C (L-Ascorbic Acid), saline, ferulic acid, hyaluronic acid (HA), and cyclosporine A.

In one example embodiment, a method for promoting eye lash growth comprises administering to a subject a composition comprising at least three compounds selected from the group consisting of platelet rich plasma (PRP), Acetylcysteine, saline, hyaluronic acid (HA), Vitamin C (L-Ascorbic Acid), ferulic acid, and cyclosporine A.

In one example embodiment, a method for treating dry eye disease comprises administering to a subject a composition comprising at least three compounds selected from the group consisting of platelet rich plasma (PRP), Acetylcysteine, saline, hyaluronic acid (HA), Vitamin C (L-Ascorbic Acid), ferulic acid, cyclosporine A, Vitamin A, Vitamin B12, Vitamin D, and Vitamin E.

In one example embodiment, a method for promoting eye lash growth comprising administering to a subject a composition comprising at least three compounds selected from the group consisting of platelet rich plasma (PRP), Acetylcysteine, saline, hyaluronic acid (HA), Vitamin C (L-Ascorbic Acid), ferulic acid, cyclosporine A, Vitamin A, Vitamin B12, Vitamin D, and Vitamin E.

In one example embodiment, a method for treating dry eye disease comprises administering to a subject a first composition comprising at least three compounds selected from the group consisting of platelet rich plasma (PRP), Acetylcysteine, saline, hyaluronic acid (HA), Vitamin C (L-Ascorbic Acid), ferulic acid, and cyclosporine A and a second composition comprising Vitamin A, Vitamin B12, Vitamin D, and Vitamin E.

In one example embodiment, a method for promoting ocular health comprises administering to a subject a composition comprising at least three compounds selected from the group consisting of Acetylcysteine, saline, hyaluronic acid (HA), Vitamin C (L-Ascorbic Acid), ferulic acid, Vitamin A, Vitamin B12, Vitamin D, and Vitamin E.

In one example embodiment, a method for promoting eye health comprises administering to a subject a composition comprising at least three compounds selected from the group consisting of Acetylcysteine, Vitamin C (L-Ascorbic Acid), ferulic acid, hyaluronic acid, and cyclosporine A.

In one example embodiment, a method for promoting eye health comprises administering to a subject a composition comprising at least three compounds selected from the group consisting of Acetylcysteine, Vitamin C (L-Ascorbic Acid), ferulic acid, hyaluronic acid, cyclosporine A, Vitamin A, Vitamin B12, Vitamin D, and Vitamin E.

In one example embodiment, a method for promoting eye health comprises administering to a subject a first composition comprising at least three compounds selected from the group consisting of Acetylcysteine, Vitamin C (L-Ascorbic Acid), ferulic acid, hyaluronic acid, and cyclosporine A and a second composition comprising Vitamin A, Vitamin B12, Vitamin D, and Vitamin E.

In one example embodiment, a method for promoting ocular health comprises administering to a subject a composition comprising at least three compounds selected from the group consisting of Acetylcysteine, Vitamin C (L-Ascorbic Acid), ferulic acid, hyaluronic acid, Vitamin A, Vitamin B12, Vitamin D, and Vitamin E.

### Dosage Forms and Routes of Administration

In some embodiments, the compositions described herein can be provided in a dosage form. The dosage form can be administered to a subject in need thereof. The dosage form can be any effective concentration administered at a given site in the subject in need thereof. As used herein, “dose,” “unit dose,” or “dosage” can refer to physically discrete units suitable for use in a subject, each unit containing a predetermined quantity of a primary composition, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism. The dosage forms can be adapted for administration by any appropriate route.

Appropriate routes include, but are not limited to, eye drops in a liquid or semi-viscous form administered via a syringe, cannula, eye drop applicator, or other suitable instrument. Other appropriate routes are described elsewhere herein. Such formulations can be prepared by any method known in the art.

Further embodiments are illustrated in the following Examples which are given for illustrative purposes only and are not intended to limit the scope of the invention.

### EXAMPLES

### Example 1—Eye Drops for Treatment of Dry Eye Disease and for Promotion of Eye Lash Growth

The compositions for treatment of DED and for promotion of eyelash growth are a unique combination of PRP, hyaluronic acid (HA), and Vitamin C. Optionally, Acetylcysteine, saline, Vitamin B12, Cyclosporine, and ferulic acid may be added. All of these ingredients have been used therapeutically for various ocular surface issues and have proven to be safe, but never have these specific ingredients been combined to form an eye drop to be used for Dry Eyes, or any other ocular surface disease.

To the Applicant's knowledge, none of these ingredients have ever been used in an eye drop designed for eyelash growth. There are many “Lash serums” on the market, but efficacy varies, and many preparations cause irritation and inflammation to the eye. The lash preparation described herein does not cause irritation and is the only lash serum on the market that is beneficial to eye health.

PRP injections have been used for treatment of dry eye disease outside of the United States, including Meibomian Gland Dysfunction, a common cause of dry eyes due to evaporation of the tear film. Published reports of PRP injection into the meibomian glands in the lower lids and/or the area of the lacrimal gland in the upper outer orbital rim, showed improvement of dry eye symptoms. These studies used a needle for the injection.

The Applicant proposes a unique technique using a cannula instead of a needle, a technique that has not been reported for the use of PRP injections for dry eye, to the Applicant's knowledge. A cannula is similar to a needle but it is not sharp so there is less tissue trauma, which is critical to preserving the integrity of the already fragile meibomian glands in dry eye patients. Cannula injections are common with cosmetic dermal filler injections and is becoming the preferred method by many cosmetic injectors. The use of the blunt cannula instead of a needle for PRP decreases trauma to the tissue to maintain the structural integrity of the meibomian glands, has a greater safety margin, less discomfort, and less risk of bruising the patient. In some cases, if the cannula does not pass without resistance subcutaneously, a needle may be necessary, as an adjunct to the cannula technique. The injector must be experienced in the use of cannulas and injections near the eye, and preferably should be performed by an Ophthalmologist.

The injection of the meibomian glands and the injection of the lacrimal glands were discussed in two separate studies conducted outside of the United States. A unique aspect of the injection described in the present application, in addition to the cannula technique, is the combination of the injection into both meibomian glands and the lacrimal gland simultaneously in one procedure, which has not been reported in the literature.

### Components of the Eye Drop and Lash Treatment Composition

### Platelet Rich Plasma (PRP)

The components of blood include red and white blood cells, platelets, and plasma. The Platelet Rich Plasma (PRP) process involves collecting a whole blood sample and centrifuging the sample to remove the red and white blood cells from the plasma, while maintaining the platelets in the plasma. There are multiple PRP kits on the market that vary in potency and purity. The potency is the percentage of platelet cells that are maintained in the plasma, and the purity is the percentage of red and white blood cells that are removed from the plasma. PRP collection kits may vary in potency and purity of the PRP. While Applicants used the Cellenis PRP collection kit (https://cellenis.com/cellenis-prp/), any appropriate PRP collection kit may be utilized to the same effect.

In practice, the subject signs a consent for the blood draw and PRP processing, and a medical history for dry eyes is taken. Parameters to follow progress include: OSDI (Ocular Surface Disease Index) survey (graded symptom questionnaire, @ Allergan, Inc.), photos, and a tear lab (osmolality) test. The OSDI overall and subscale scores range from 0 to 100. Based on their OSDI scores, patients can be categorized as having a normal ocular surface (0-12 points) or as having mild (13-22 points), moderate (23-32 points), or severe (33-100 points) ocular surface disease. The scoring of the OSDI was performed according to the published guidelines. The OSDI has satisfactory internal consistency, test-retest reliability, validity, sensitivity, and specificity for use among patients with ocular surface disease, and is a parameter commonly used in Ophthalmology research. A sample of 11cc of blood, or more depending on the PRP processing kit being used, is drawn into the PRP collection tube with an anticoagulant, then centrifuged for 1-10 minutes, depending on the system used. The PRP is collected from the tube in a sterile 20cc syringe and set aside. The volume of PRP collected is usually 6-8 cc. This pure PRP can be used for injection into the tissue around the eye to treat dry eyes.

### Acetylcysteine (a Precursor to Glutathione)

A 20% Sterile Preservative free Acetylcysteine solution made for injection may be used and titrated to the appropriate dilution for optimal comfort and physiologic pH.

### Vitamin C (L-Ascorbic Acid)

A 50% solution of Preservative Free, Sterile Vitamin C, made for injection, is used and titrated to the appropriate dilution for optimal comfort and physiologic pH. Again, the use of Vitamin C is optional and offered by way of example and not limitation.

### Ferulic Acid

As Vitamin C may destabilize fairly quickly when used in solution, a 0.05% to 0.5% solution of ferulic acid was used as a Vitamin C stabilizer, optionally dissolved in propylene glycol. The use of ferulic acid is optional and offered by way of example and not limitation.

### Vitamin B12

A preservative-free Vitamin B12 eye drop solution was used and can be obtained from any compound pharmacy. In Applicant's example, an effective amount of Vitamin B12 may be added as set forth in Table 1. The use of Vitamin B12 is optional and offered by way of example and not limitation.

### Hyaluronic Acid (HA)

Hyaluronic acid (HA) is a natural substance found in the fluids in the eyes, skin, and joints. It acts as a cushion and lubricant in the joints and other tissues. Different forms of hyaluronic acid are used for cosmetic purposes. Hyaluronic acid might also affect the way the body responds to injury and help to decrease swelling. Hyaluronic acid can be taken by mouth and or applied to the skin for UTIs, acid reflux, wound healing, aging skin, and many other conditions. Applicant contemplates inclusion of HA in the composition for treatment of dry eye and to promote lash growth; HA is naturally secreted by the corneal epithelial cells and is an excellent lubricant. HA is often used as an active ingredient in many artificial tear eye drops that are currently commercially available. The presence of an antioxidant may enhance the production of endogenous hyaluronic acid and decrease the breakdown of hyaluronic acid, optimizing the hyaluronic acid on the ocular surface.

### Cyclosporine A

A commercially available generic preparation of preservative free 0.05% Cyclosporine eye drops was used. Cyclosporine is contemplated as a possible compound for inclusion in the eye drop composition, if the composition were to be a prescription-based product. It is also contemplated that the cyclosporine may be omitted so that the result composition may be obtained as an over-the-counter product.

### Formula, Preparation, and Protocol

To prepare the eye drops and lash preparation, the pure PRP is combined with possibly Acetylcysteine, possibly Vitamin C, possibly ferulic acid, possibly Vitamin B12 and possibly Cyclosporine. Estimated amounts of each compound are shown in Table 1. The Applicant tested the pH of the solutions to see what combination creates a pH that is most physiologic to the pH of human tears. These ingredients are added to the sterile 20cc syringe containing the PRP. Preservative free saline or artificial tears containing hyaluronic acid (HA), were then added to this solution to bring the total volume to about 15 cc, resulting in a solution that is approximately 50% PRP.

The syringe is gently inverted to mix the ingredients and is transferred to 12 sterile 3cc eyedropper bottles, under a laminar flow hood with a HEPA filter. The process results in at least 1.25 cc in each of the 12 eyedropper bottles. The patient is instructed to use one bottle at a time, and to store the others in a freezer until needed. They are instructed to use the drops at least four times daily in each eye for 2 weeks, and then may titrate the dose up or down, depending on the severity of the dry eyes, but to maintain twice daily as a maintenance dose. For lash growth, one drop twice daily is recommended.

Applicant investigated various combinations and concentrations using the above mentioned components. Applicant manufactured the eye drop composition and treated patients with the following formula: 1) Platelet Rich Plasma: The applicant obtained commercially available PRP kits made by companies with the intention to be used for PRP injections in other fields including Orthopedics, Dermatology, and Cosmetic procedures. Any commercially available kit can be used for this purpose with appropriate purity and potency; 2) Vitamin C (Ascor) 50% sterile solution made for injection, obtained from a medical supply company, shelf life 18 months; 3) Preservative Free Artificial Tears containing hyaluronic acid.

Using aseptic technique under a laminar flow hood, the Vitamin C was diluted with the artificial tears containing hyaluronic acid and titrated to a concentration that was consistent with patient comfort and a physiologic pH, while maintaining an optimal concentration of the active ingredients, and keeping in mind the increased corneal sensitivity in patients with dry eyes.

Volunteer subjects were asked to grade the comfort of the drops on a scale of 0-5, 0 being no discomfort, and 5 being maximum discomfort. The solution was diluted to a concentration in which the comfort scored 0.5 on a scale of 0-5, reflecting minimal corneal sensitivity.

The final pH of the solution was 6.75, which is physiologic with the eye. The final concentration of the Vitamin C was 5%, and the sensitivity score was 0.5 on a scale of 0-5, with minimal sensitivity after instillation of the drop.

The components and preparation for the lash treatment composition are identical to the eye drop composition, except the intended goal is to enhance eyelash growth. The dosage is 2 drops daily.

### Example 2—Eye Shot with Platelet Rich Plasma (PRP) Only

In another aspect, 100% pure PRP is administered to a subject in need to treat dry eye disease. As described above, after the periorbital skin has been cleansed with surgical grade Betadine wipes, a 23-gauge needle is used to create a micro-port in the skin near the lateral canthus. A 25-gauge cannula is attached to the syringe containing the pure PRP, and the blunt tip of the cannula is inserted into the port created by the needle. The cannula slides under the skin along the lower lid anterior to the tarsal plate in the region of the meibomian glands and the PRP is delivered as the cannula is withdrawn. The cannula is then directed to the meibomian glands along the upper lid margin, and to the outer orbital rim in the region of the lacrimal gland. In some cases, if the cannula doesn't pass without resistance subcutaneously, a needle may be necessary, as an adjunct to the cannula technique. Approximately 1cc of PRP is delivered through the cannula to each area, in both eyes, injecting approximately 6 cc of PRP total.

### Formula, Preparation, and Protocol

The intended goal of the PRP shot is tissue regeneration in the Meibomian Glands of the upper and lower lids and the Lacrimal Gland in the upper outer orbit. The intended patient population target is patients with moderate to severe dry eyes in which other therapies have not proven effective. Risks involve infection, edema, bruising, and rarely, injury to the eye.

The patient signs an informed consent. The PRP is prepared as described above using the commercially available PRP kits intended for other fields, such as Orthopedics, Dermatology, and/or cosmetic procedures.

The 6-8cc of processed PRP is used as an injection without dilution, i.e. 100% PRP.

The periorbital area is prepped with surgical grade betadine wipes, and a topical anesthetic is applied to the temporal orbital rims.

Using a cannula technique with the entry site near the lateral canthus, 1cc of PRP is injected subcutaneously into each of the following areas: the meibomian gland region of the lower lid, the meibomian gland region of the upper lid, and to the lacrimal gland area just below the orbital rim in the outer ⅓ of the orbit. The cannula technique decreases the risk of complications, tissue trauma, pain, and bruising, compared to the needle technique. A 25 g cannula is used with a 23 g needle for the entry site. In some cases, if the cannula doesn't pass easily due to resistance, a needle may be used to cautiously deliver the PRP.

The patient will have boggy eyelids due to the PRP volume, but this resolves over a period of minutes with gentle massage.

TABLE 1
Concentration Ranges for an Example Composition for Treatment
of Dry Eye Disease and Promoting Lash Growth

| | Lower | Target | Upper |
|---|---|---|---|
| | Concentration | Concentration | Concentration |
| Compound | Value | Value | Value |
| Platelet Rich | Drops: 20% | 50% | 70% |
| Plasma (PRP) | Shot: 100% | Shot: 100% | Shot 100% |
| Ferulic Acid | Drops: 1 drop/ | Drops: 1 drop/ | Drops: 3 drops/ |
| (stabilizer for | 5 cc, .05% | 5 cc, .1% | 5 cc, .5% |
| Vitamin C) | solution | solution | solution |
| Acetylcysteine | Drops: 3% | Drops: 3.3% | Drops: 10% |
| Vitamin C | Drops: 1% | Drops: 8.3% | Drops: 15% |
| (L-Ascorbic Acid) | | | |
| Cyclosporine A | Drops: .05% | Drops: .05% | Drops: .10% |
| Hyaluronic Acid | Drops: .15% | Drops: .3% | Drop: .5% |
| Vitamin B12 | Drops: .02% | Drops: .04% | Drops: .05% |
| Vitamin A | Drops: .01% | Drops: .05% | Drops: .05% |

### Clinical Experience/Data for Examples 1 and 2

### Eye Drop Composition for DED

### Patient Demographics

Applicant provided the eye drop composition to 43 patients using the protocol described above. 93% of the patients were female, and 7% were men. Average age was 56 yrs old.

### Adverse Effects

One patient reported stinging with the eyedrops and discontinued the drops after 2 weeks. Otherwise, there were no adverse effects reported.

### Results

The endpoint parameter with the most validity and practicality in a practice setting proved to be the OSDI (Ocular Surface Disease Index) Survey score, which is based on patients' symptoms. Other parameters proved to be less reliable, including tear osmolality and photos. The OSDI survey, developed and copyrighted by Allergan, Inc, is a 12-item self-success. It is well recognized in the field of Dry Eye research that the OSDI has satisfactory internal consistency, test-retest reliability, validity, sensitivity, and specificity for use among patients with ocular surface disease.

The OSDI scores range from 0 to 100. Based on the OSDI scores, dry eye severity can be categorized:
0-12: Normal, no dry eye
13-22: Mild Dry Eye
23-32: Moderate Dry Eye
33-100: Severe Dry Eye

The following results are based on 26 patient who completed an OSDI survey after treatment. The patient numbers and average OSDI scores in each category are as follows:

Administered questionnaire, and has been used successfully by researchers and clinicians to assess treatment

TABLE 2
OSDI Survey After Treatment

| | | | Average OSDI |
|---|---|---|---|
| | | | post-treatment/% |
| | | Average OSDI | of pts with |
| DRY EYE severity | #of patients | pre-treatment | improved score |
| 0-12: No Dry Eye. | 3 patients | 9 | 4 100% of |
| Patients using | | | pts improved |
| drops for lashes | | | |
| 13-22: Mild | 8 patients | 16 | 6 100% of |
| Dry Eye | | | pts improved |
| 23-32: Moderate | 8 patients | 27 | 18 88% of |
| Dry Eye | | | pts improved |
| 33-100: Severe | 7 patients | 37 | 25 86% of |
| Dry Eye | | | pts improved |
| Total: | 26 patients | 24 | 15 92% of |
| | | | pts improved |

### Patient Satisfaction:

96% of the patients who completed at least one draw (3-6 months) of the eyedrops reported they would like to continue using the drops. The 2 patients who did not want to continue cited cost as being prohibitive. None of these patients reported adverse events.

### Treatment for Lash Growth

Four patients in the group described above used the drops for the purpose of Eyelash enhancement. Amongst these four patients, 100% noticed improvement of lashes in length and thickness. Amongst total patients, 46% noticed lash improvement. It should be noted that 85% of this group were using the drops for the purpose of dry eyes and may not have noticed a change in the eyelashes as it was less of a priority than the improvement of their dry eye symptoms.

Eye Shot with Platelet Rich Plasma (PRP) Only

### Patient Demographics:

Two patients opted to have the shot, i.e. PRP injections in the meibomian gland and lacrimal gland regions. Both patients were Asian females, ages 28 and 54.

### Treatment

Both patients received a 3cc injection around each eye at each session. 3 sessions were done about 1 month apart, totaling 3 injections around each eye over a 3 month period.

### Results

Both patients showed an improvement in their OSDI scores: OSDI 29 improved to 10 OSDI 34 improved to 31.

### Adverse Effects

Both patients experienced boggy eyelids from the injected PRP volume that resolved completely over several hours as the PRP was absorbed into the tissue. Both patients had occasional pinpoint bruising at the entry site of the injection. Otherwise, there were no adverse events.

### Example 3—Eye Drops for Promoting Ocular Health and Prevention of Oxidative Damage to the Eye

Applicant evaluated the benefit of adding other Vitamins to the eye drops, described above, including, but not limited to, Vitamin A, and Vitamin B12. Applicant also evaluated a nutritional supplement eye drop to use with the PRP eye drops or as a stand-alone nutritional supplement. The nutritional supplement may contain only the vitamins and not the PRP, that is, a supplement eye drop containing Acetylcysteine, Vitamin C, Vitamin A, and Vitamin B12, and possibly other ingredients, to maintain the health of the ocular surface. The evidence for this is discussed above.

Vitamin A and Vitamin B12 eye drops are both available through compound pharmacies in solutions that are appropriate for the eye. Applicant evaluated the benefit of adding these to eye drops containing the PRP and other compounds discussed above. As noted above, Applicant further created a stand-alone nutritional eye drop, which only contains the vitamins and not the PRP. That is, a supplement eye drop containing at least three compounds selected from Acetylcysteine, hyaluronic acid (HA), Vitamin C, Vitamin A, and Vitamin 12, and ferulic acid, to maintain the health of the ocular Surface.

The compositions for promoting ocular health are a unique combination of Acetylcysteine, Vitamin C, and hyaluronic acid. Saline, stabilizer such as ferulic acid, Vitamin B12 and/or Cyclosporine are all further optional ingredients. All of these ingredients have been used for various ocular surface issues and have proven to be safe but never have these specific ingredients or any combination of these specific ingredients been combined to form an eye drop to be used to promote eye health, or any ocular surface disease.

To the Applicant's knowledge, none of these ingredients have ever been used in an eye drop designed for eye health, that is, as a nutritional supplement specifically to prevent oxidative damage due to UV light, essentially a topical sunscreen for the eye. This vitamin supplement eye drop may contain Acetylcysteine, Vitamin C, Vitamin A, and Vitamin B12, and possibly other ingredients, to maintain the health of the ocular surface. The evidence for this is discussed above.

### Components

### Acetylcysteine (a Precursor to Glutathione)

A 20% Sterile Preservative free Acetylcysteine solution made for injection is used and titrated to the appropriate dilution for optimal comfort and physiologic pH.

### Vitamin C (L-Ascorbic Acid)

A 50% solution of Preservative Free, Sterile Vitamin C, made for injection, is used and titrated to the appropriate dilution for optimal comfort and physiologic pH. Again, the use of Vitamin C is optional and offered by way of example and not limitation.

### Ferulic Acid

As Vitamin C may destabilize fairly quickly when used in solution, a 0.05% to 0.5% solution of ferulic acid was used as a Vitamin C stabilizer. The use of ferulic acid is optional and offered by way of example and not limitation.

### Vitamin B12

A preservative-free Vitamin B12 eye drop solution can be used and can be obtained from a compound pharmacy. In Applicant's example, an effective amount of Vitamin B12 may be added as set forth in Table 3. The use Vitamin B12 is optional and offered by way of example and not limitation.

### Hyaluronic Acid (HA)

Hyaluronic acid (HA) is a natural substance found in the fluids in the eyes, skin, and joints. It acts as a cushion and lubricant in the joints and other tissues. Different forms of hyaluronic acid are used for cosmetic purposes. Hyaluronic acid might also affect the way the body responds to injury and help to decrease swelling. Hyaluronic acid can be taken by mouth and or applied to the skin for UTIs, acid reflux, wound healing, aging skin, and many other conditions. Applicant contemplates inclusion of HA in the composition to promote ocular health and comfort; HA is naturally secreted by the corneal epithelial cells and is an excellent lubricant. HA is often used as an active ingredient in many artificial tears drops that are currently commercially available. The presence of an antioxidant may enhance the production of endogenous hyaluronic acid and decrease the breakdown of Hyaluronic acid, optimizing the hyaluronic acid on the ocular surface.

### Cyclosporine A

A commercially available generic preparation of preservative free 0.05% Cyclosporine eye drops was used. Cyclosporine is contemplated as a possible compound for inclusion in the eye drop composition, if the composition were to be a prescription-based product. It is also contemplated that the cyclosporine may be omitted so that the result composition may be obtained as an over-the-counter product.

### Eye Drop Preparation.

To prepare the eye drops, the following components are combined: possibly Acetylcysteine, possibly Vitamin C, possibly ferulic acid, possibly Vitamin B12, possibly Vitamin A, and possibly Cyclosporine. Estimated amounts of each compound are shown in Tables land 3. The Applicant tested the pH of the solutions to see what combination creates a pH that is most physiologic to the pH of Human tears. The ingredients are combined in a sterile 30 cc bottle, under a laminar flow hood with a HEPA filter. Preservative free saline or preservative free artificial tears containing hyaluronic acid are then added to this solution to bring the total volume to about 30 cc. The solution has a physiologic pH.

The bottle is gently inverted to mix the ingredients and using a 10 cc syringe, 5 cc is transferred to 6 sterile glass eyedropper bottles, under a laminar flow hood with a HEPA filter. The bottles are capped with a sterile AeroPump cap using a press to seal the cap, creating 5 cc of the product in each Preservative Free MultiDose bottle. The AeroPump cap is dose metered to deliver a 28 microliter drop. The patient is instructed to use one drop one to two times daily to promote ocular health.

TABLE 3
Concentration Ranges for Nutritional Supplement Eyedrop

| | Lower | Target | Upper |
|---|---|---|---|
| | Concentration | Concentration | Concentration |
| Compound | Value | Value | Value |
| Vitamin A | .01% | .05% | .05% |
| Vitamin B12 | .02% | .05% | .3% |
| Vitamin D | .02% | .04% | .05% |
| Vitamin E | 1% | 1% | 2% |
| Hyaluronic Acid | .15% | .3% | .5% |
| Vitamin C | 1% | 8.3% | 20% |
| (L-Ascorbic Acid) | | | |
| Ferulic Acid | 1 drop/5 cc, | 1 drop/5 cc, | 3 drop/5 cc, |
| (stabilizer for | .05% solution | .1% solution | .5% solution |
| Vitamin C) | | | |
| Acetylcysteine | 3% | 3.3% | 20% |
| Cyclosporine A | .05% | .05% | .10% |

### Vitamin SEE Formula and Preparation:

The proposed vitamin supplement eyedrop to promote ocular health has the following formula:

### Initial Components:

Acetylcysteine 20% sterile solution for injection, obtained from a medical supply company, shelf life 18 months.

Vitamin C (Ascor) 50% sterile solution made for injection, obtained from a medical supply company, shelf life 18 months.

Preservative free Artificial tears containing hyaluronic acid, optional ferulic acid.

### Preparation

Using aseptic technique under a laminar flow hood, Acetylcysteine and Vitamin C were diluted with the artificial tears containing hyaluronic acid and titrated to a concentration that was consistent with patient comfort and a physiologic pH, while maintaining an optimal concentration of the active ingredients.

Volunteer subjects were asked to grade the comfort of the drops on a scale of 0-5, 0 being no discomfort, and 5 being maximum discomfort. The drops were diluted to a concentration in which the comfort scored at 2 on a scale of 0-5, so only a slight burning sensation for a few seconds after instillation of the drop. Approximately 100 bottles have been distributed to patients with no reported adverse events.

The final pH of the eyedrop solution was 6.5, which is physiologic with the eye.

The final concentrations of the components were 3.3% Acetylcysteine; 8.3% Vitamin C, in a solution of preservative free artificial tears containing hyaluronic acid, and optional Ferulic acid.

Final formula: 1 part AC, 1 part Vit C, and 4 parts Systane Hydration:=3.3% AC, 8.3% Vit C (See math worksheet below) pH=6.

Mathematical breakdown of final formula:

$$1 part AC / 1 part VitC / 4 parts HA artificial tears :$$

$$Stock AC Solution : 20 \% AC = 200 mg / ml$$

$$1 ml diluted to 6 ml total = 200 mg / 6 cc = 3.3 \% AC$$

$$Stock VIT C Solution : 50 \% VitC = 500 mg / ml$$

$$1 ml diluted to 6 ml total = 500 mg / 6 cc = 8.3 \% Vit C$$

$$3.3 \% AC / 8.3 \% Vit C in HA artificial tears , pH = 6.5$$

Formula for 30 cc of Vitamin eye drops: 5 cc AC+5 cc VitC+20 ccAT (HA artificial tear)=30 cc Vitamin SEE (3.3% AC, 8.3% Vitamin C).

### Protocol for Vitamin SEE Preparation:

All steps are done using aseptic technique under a Laminar flow hood with a HEPA filter:
1. Add 5 cc of a 50% Vit C solution (500 mg/cc) to an empty sterile 30 cc bottle.
2. Add 5 cc of a 20% Acetylcysteine solution (200 mg/cc) to the same bottle.
3. Set the bottle aside.
4. Remove the plunger from a 20 cc syringe, to be used as a sterile measuring tube. With a 27 g needle on the syringe to prevent leakage, turn the syringe upside down with the open end up, and put it in the test tube holder under the hood.
5. Add 20 cc of Artificial Tears with hyaluronic acid.
6. Add the 20 cc of HA Artificial tears to the bottle containing the Vit C and Acetylcysteine, to create 30 cc total of Vit SEE drops.
7. Shake well.
8. Using the sterile 3K Preservative-free multidose eyedropper bottles made by AeroPump, add 5 cc to each bottle and cap with a sterile cap using a press to seal, and label.
9. Add one drop, 0.05% to 0.5% ferulic acid solution, optional.
10. Each bottle contains 5 cc of Vitamin SEE: 3.3% Acetylcysteine, 8.3% Vitamin C in hyaluronic acid. Preservative free in a multidose bottle.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

### REFERENCES

1. Alio J L, Rodriguez A E, Ferreira-Oliveira R, Wróbel-Dudzińska D, Abdelghany A A. Treatment of dry eye disease with autologous platelet-rich plasma: a prospective, interventional, non-randomized study. Ophthalmol Ther. 2017; 6(2):285-293. doi: 10.1007/s40123-017-0100-z
2. Murtaza F, Toameh D, Chiu H H, Tam E S, Somani S. Autologous Platelet-Rich Plasma Drops for Evaporative Dry Eye Disease from Meibomian Gland Dysfunction: A Pilot Study. Clin Ophthalmol. 2022 Jul. 6; 16:2199-2208. doi: 10.2147/OPTH.S367807.PMID: 35821787; PMCID: PMC9271282.
3. Mohammed, M. A., Allam, I. Y., Shaheen, M. S. et al. Lacrimal gland injection of platelet rich plasma for treatment of severe dry eye: a comparative clinical study. BMC Ophthalmol 22, 343 (2022). https://doi.org/10.1186/s12886-022-02554-0
4. Avila M Y, Igua A M, Mora A M. Randomised, prospective clinical trial of platelet-rich plasma injection in the management of severe dry eye British Journal of Ophthalmology 2019; 103:648-653.
5. Mohammad, E., Lamie, N., Ismail, A., Mansour, M. (2022). ‘Autologous Platelet Rich Plasma versus Autologous Serum Eye Drops in Treatment of Severe Dry Eye Disease’, International Journal of Medical Arts, 4(8), pp. 2542-2549. doi: 10.21608/ijma.2022.152958.1490
6. Li Z J, Choi H I, Choi D K, et al. Autologous platelet-rich plasma: a potential therapeutic tool for promoting hair growth. Dermatol Surg. 2012; 38(7 Pt 1):1040-1046. doi: 10.1111/j.1524-4725.2012.02394
7. Law S K. Bimatoprost in the treatment of eyelash hypotrichosis. Clin Ophthalmol. 2010 Apr. 26; 4:349-58. doi: 10.2147/opth.s6480. PMID: 20463804; PMCID: PMC2861943.
8. Elgindy E A, El-Huseiny A M, Mostafa M I, Gaballah A M, Ahmed T A. N-acetyl cysteine: could it be an effective adjuvant therapy in ICSI cycles. A preliminary study. Reprod Biomed Online. 2010; 20 (6):789-796. [PubMed] [Google Scholar]
9. Mokhtari V, Afsharian P, Shahhoseini M, Kalantar S M, Moini A. A Review on Various Uses of N-Acetyl Cysteine. Cell J. 2017 April-June; 19(1):11-17. doi: 10.22074/cellj.2016.4872. Epub 2016 Dec. 21. PMID: 28367412; PMCID: PMC5241507.
10. Ganea E, Harding J J. Glutathione-related enzymes and the eye. Curr Eye Res. 2006 January; 31(1):1-11. doi: 10.1080/02713680500477347. PMID: 16421014.
11. Yas Eghtedari, Lawrence J. Oh, Nick Di Girolamo, Stephanie L. Watson, The role of topical Acetylcysteine in ocular therapeutics, Survey of Ophthalmology, Volume 67, Issue 2, 2022, Pages 608-622, ISSN 0039-6257
12. Mahira Hamdy El Sayed, Marwa Yassin Soltan, Ahmed Sadek, Mohamed Abo Shabana Hussein Mohamed Efficacy and Tolerability of N-Acetyl-Cysteine for Treatment of The Early-onset Androgenetic Alopecia in Men QJM: An International Journal of Medicine, Volume 114, Issue Supplement_1, October 2021, hcab093.024
13. Huang J Y, Yeh P T, Hou Y C. A randomized, double-blind, placebo-controlled study of oral antioxidant supplement therapy in patients with dry eye syndrome. Clin Ophthalmol. 2016; 10:813-820
14. Ohigashi H, Hashimoto D, Hayase E, et al. Ocular instillation of vitamin A-coupled liposomes containing HSP47 siRNA ameliorates dry eye syndrome in chronic GVHD. Blood Adv. 2019; 3(7):1003-1010.
15. Al-Namaeh M, Andersson R. Effects of ascorbic acid on chemical and thermal corneal burns: A comprehensive literature review. Med Hypothesis Discov Innov Optom. 2020 Fall; 1(2): 44-51. DOI: https://doi.org/10.51329/mehdioptometry107
16. Paterson Christopher A, O'Rourke Melinda C. Vitamin C levels in human tears. Arch Ophthalmol. 1987 March; 105(3):376-7. doi: 10.1001/archopht.1987.01060030096034. PMID: 3827714.
17. Kwack M H, Shin S H, Kim S R, Im S U, Han I S, Kim M K, Kim J C, Sung Y K. 1-Ascorbic acid 2-phosphate promotes elongation of hair shafts via the secretion of insulin-like growth factor-1 from dermal papilla cells through phosphatidylinositol 3-kinase. Br J Dermatol. 2009 June; 160(6):1157-62. doi: 10.1111/j.1365-2133.2009.09108.x. Epub 2009 Mar. 26. PMID: 19416266.
18. Periman L M, Mah F S, Karpecki P M. A Review of the Mechanism of Action of Cyclosporine A: The Role of Cyclosporine A in Dry Eye Disease and Recent Formulation Developments. Clin Ophthalmol. 2020 Dec. 2; 14:4187-4200. doi: 10.2147/OPTH.S279051. PMID: 33299295; PMCID: PMC7719434.
19. Lan S, Liu F, Zhao G, Zhou T, Wu C, Kou J, Fan R, Qi X, Li Y, Jiang Y, Bai T, Li P, Liu L, Hao D, Zhang L, Li Y, Liu J Y. Cyclosporine A increases hair follicle growth by suppressing apoptosis-inducing factor nuclear translocation: a new mechanism. Fundam Clin Pharmacol. 2015 April; 29 (2): 191-203. doi: 10.1111/fcp.12100. Epub 2015 Mar. 8. PMID: 25619112.
20. Amemiya T. The eye and nutrition. Jpn J Ophthalmol. 2000 May; 44(3):320. doi: 10.1016/s0021-5155(00)00161-1. PMID: 10913665.
21. Fogagnolo P, De Cilla'S, Alkabes M, Sabella P, Rossetti L. A Review of Topical and Systemic Vitamin Supplementation in Ocular Surface Diseases. Nutrients. 2021 Jun. 10; 13(6):1998. doi: 10.3390/nu13061998. PMID: 34200595; PMCID: PMC8228525.
22. Chelala E, Dirani A, Fadlallah A, Fahd S. The role of topical vitamin A in promoting healing in surface refractive procedures: a prospective randomized controlled study. Clin Ophthalmol. 2013; 7:1913-8. doi: 10.2147/OPTH.S52280. Epub 2013 Sep. 24. PMID: 24109170; PMCID: PMC3792968.
23. Toshida H, Funaki T, Ono K, Tabuchi N, Watanabe S, Seki T, Otake H, Kato T, Ebihara N, Murakami A. Efficacy and safety of retinol palmitate ophthalmic solution in the treatment of dry eye: a Japanese Phase I I clinical trial. Drug Des Devel Ther. 2017 Jun. 23; 11:1871-1879. doi: 10.2147/DDDT.S137825. Erratum in: Drug Des Devel Ther. 2021 Feb. 24; 15:813-816. PMID: 28694687; PMCID: PMC5491700.
24. Cui X., Xiang J., Zhu W., Wei A., Le Q., Xu J., Zhou X. Vitamin A Palmitate and Carbomer Gel Protects the Conjunctiva of Patients With Long-term Prostaglandin Analogs Application. J. Glaucoma. 2016; 25:487-492.
25. Kim E. C., Choi J. S., Joo C. K. A comparison of vitamin a and cyclosporine a 0.05% eye drops for treatment of dry eye syndrome. Am. J. Ophthalmol. 2009; 147:206-213.e3. doi: 10.1016/j.ajo.2008.08.015.
26. Selek H., Unlü N., Orhan M., Irkeç M. Evaluation of retinoic acid ophthalmic emulsion in dry eye. Eur. J. Ophthalmol. 2000; 10:121-127.
27. Soong H. K., Martin N. F., Wagoner M. D., Alfonso E., Mandelbaum S. H., Laibson P. R., Smith R. E., Udell I. Topical retinoid therapy for squamous metaplasia of various ocular surface disorders. A multicenter, placebo-controlled double-masked study. Ophthalmology. 1988; 95:1442-1446.
28. Tseng S. C., Maumenee A. E., Stark W. J., Maumenee I. H., Jensen A. D., Green W. R., Kenyon K. R. Topical retinoid treatment for various dry-eye disorders. Ophthalmology. 1985; 92:717-727.
29. Yang J., Liu Y., Xu Y., Li X., Fu J., Jiang X., Chou Y., Ma J., Hao R., Zhang R., et al. A new approach of ocular nebulization with vitamin B12 versus oxytocin for the treatment of dry eye disease: An in vivo confocal microscopy study. Drug Des. Devel. Ther. 2019; 13:2381-2391
30. Fogagnolo P., Melardi E., Tranchina L., Rossetti L. Topical citicoline and vitamin B12 versus placebo in the treatment of diabetes-related corneal nerve damage: A randomized double-blind controlled trial. BMC Ophthalmol. 2020; 20:315.
31. Macri A., Scanarotti C., Bassi A. M., Giuffrida S., Sangalli G., Traverso C. E., Iester M. Evaluation of oxidative stress levels in the conjunctival epithelium of patients with or without dry eye, and dry eye patients treated with preservative-free hyaluronic acid 0.15% and vitamin B12 eye drops. Graefes. Arch. Clin. Exp. Ophthalmol. 2015; 253:425-430.
32. Lundström I. M., Lindström F. D. Iron and vitamin deficiencies, endocrine and immune status in patients with primary Sjögren's syndrome. Oral. Dis. 2001; 7:144-149.
33. Andrès E., Blicklé F., Sordet C., Cohen-Solal J., Sibilia J., Sapin R. Primary Sjögren's syndrome and vitamin B12 deficiency: Preliminary results in 80 patients. Am. J. Med. 2006; 119: e9-e10
34. Maria Rosaria Romano, Effects of vitamin B12 on the corneal nerve regeneration in rats, Experimental Eye Research, Volume 120,2014, Pages 109-117.
35. De-Hita-Cantalejo C, Sánchez-González M C, Silva-Viguera C, García-Romera M C, Feria-Mantero R, Sánchez-González J M. Efficacy of hyaluronic acid 0.3%, cyanocobalamin, electrolytes, and P-Plus in menopause patients with moderate dry eye disease. Graefes Arch Clin Exp Ophthalmol. 2022 February; 260 (2): 529-535.
36. Shetty R, Deshpande K, Ghosh A, Sethu S. Management of Ocular Neuropathic Pain With Vitamin B12 Supplements: A Case Report. Cornea. 2015 October; 34 (10): 1324-5. doi: 10.1097/ICO.0000000000000572. PMID: 26266431.
37. Clinical observation of vitamin B12 eye drops for vision fatigue caused by visual display terminals Qiang Guo; Hong-Bin Yang; Zhuo-Lei Feng Source: Guoji Yanke Zazhi, Vol 16, Number 7, 2016, pp. 1329-1331 (3) Publisher: Directory of Open Access Journals.

### REFERENCES FOR FERULIC ACID STABILIZATION OF VITAMIN C

38. Yani Wang, Zhaoyang Wang, Tengteng Yao, et al. The Potential Values of Ferulic Acid in Ophthalmic Diseases. Authorea. Apr. 28, 2023.

DOI: 10.22541/au.168268223.36762307/v1
39. Chen H C, Chen Z Y, Wang T J, Drew V J, Tseng C L, Fang H W, Lin F H. Herbal Supplement in a Buffer for Dry Eye Syndrome Treatment. Int J Mol Sci. 2017 Aug. 3; 18 (8): 1697. doi: 10.3390/ijms18081697. PMID: 28771187; PMCID: PMC5578087.
40. Chen Y, Zhu L, Meng H, Sun X, Xue C. Ferulic Acid Protects Human Lens Epithelial Cells against Ionizing Radiation-Induced Oxidative Damage by Activating Nrf2/HO-1 Signal Pathway. Oxid Med Cell Longev. 2022 May 10; 2022:6932188. doi: 10.1155/2022/6932188. PMID: 35592532; PMCID: PMC9113866.
41. Hsiung Lin, Jing-Yi Lin, Ravindra D. Gupta, Joshua A. Tournas, James A. Burch, M. Angelica Selim, Nancy A. Monteiro-Riviere, James M. Grichnik, Jan Zielinski, Sheldon R. Pinnell, Ferulic Acid Stabilizes a Solution of Vitamins C and E and Doubles its Photoprotection of Skin, Journal of Investigative Dermatology, Vol 125, Issue 4, October 2005, pages 826-832
42. Feng, Yangyang Zhang * The potential benefits of polyphenols for corneal diseases Biomedicine & Pharmacotherapy 169 (2023) 115862
43. G, Jiang, T. Fan Sodium Ferulate Attenuates Lidocaine-Induced Corneal Endothelial Impairment, Oxid. Med Cell Lonbev. Vol 2018 (2018) 4967398
44. C.-Y. Tsai, et al., Thermosensitive chitosan-based hydrogels for sustained release of ferulic acid on corneal wound healing, Carbohydr. Polym. vol. 135 (2016) 308-315, https://doi.org/10.1016/j.carbpol.20015.08.098.
45. Dolińska B, Ostróżka-Cieślik A, Caban A, Rimantas K, Leszczyńska L, Ryszka F. Influence of trace elements on stabilization of aqueous solutions of ascorbic acid. Biol Trace Elem Res. 2012 December; 150 (1-3): 509-12. doi: 10.1007/s12011-012-9524-4. Epub 2012 Oct. 26. PMID: 23099563; PMCID: PMC3510389.
46. Swindle-Reilly, Katelyn, et al. Methods to Improve Vitamin C Stability in Hydrogel Vitreous Substitutes to Prevent Cataract, Investigative Ophthalmology & Visual Science June 2020, Vol 61, 3726

What is claimed is:

1. A composition for treating dry eye disease and promoting eye lash growth, comprising at least three compounds selected from the group consisting of platelet rich plasma (PRP), Acetylcysteine, Vitamin C (L-Ascorbic Acid), cyclosporine A, and hyaluronic acid (HA).

2. The composition of claim 1, further comprising a ferulic acid solution.

3. The composition of claim 2, wherein the ferulic acid solution comprises 0.05% to 0.5% ferulic acid.

4. The composition of claim 1, further comprising at least one of Vitamin B12, Vitamin D, Vitamin A, and Vitamin E.

5. A composition for promoting ocular health, comprising at least three compounds selected from the group consisting of Acetylcysteine, hyaluronic acid, Vitamin C (L-Ascorbic Acid), Vitamin A, Vitamin B12, Vitamin D, and Vitamin E.

6. The composition of claim 5, further comprising a ferulic acid solution.

7. The composition of claim 6, wherein the ferulic acid solution comprises 0.05% to 0.5% ferulic acid.

8. A composition for treating dry eyes comprising 100% pure platelet rich plasma (PRP).

9. A method for treating dry eye disease and promoting eye lash growth, comprising administering to a subject a composition comprising at least three compounds selected from the group consisting of platelet rich plasma (PRP), Acetylcysteine, Vitamin C (L-Ascorbic Acid), hyaluronic acid (HA), Vitamin A, and cyclosporine A.

10. The method of claim 9, further comprising administering ferulic acid.

11. The method of claim 9, further comprising administering at least one of Vitamin B12, Vitamin D, Vitamin E.

12. A method for promoting ocular health comprising administering to a subject a composition comprising at least three compounds selected from the group consisting of Acetylcysteine, hyaluronic acid (HA), Vitamin C (L-Ascorbic Acid), Vitamin A, Vitamin B12, Vitamin D, and Vitamin E.

13. The method of claim 12, further comprising administering a ferulic acid solution.

14. The method of claim 13, wherein the ferulic acid solution comprises 0.05% to 0.5% ferulic acid.

15. A method for treating dry eye disease comprising administering to a subject a composition comprising 100% pure platelet rich plasms (PRP).
